(19) United States
(12) Patent Application Publication
(10) Pub. No.: US 2003/0045771 A1
(43) Pub. Date: Mar. 6, 2003

(54) Heart wall tension reduction devices and methods

(76) Inventors: Cyril J. Schweich JR., Maple Grove, MN (US); Todd J. Mortier, Minneapolis, MN (US); Robert M. Vidlund, Maplewood, MN (US); Thomas M. Paulson, Minneapolis, MN (US); Peter T. Keith, St. Paul, MN (US); David A. Kusz, Minneapolis, MN (US)

Correspondence Address: Finnegan, Henderson, Farabow,, Garrett & Dunner, L.L.P., 1300 I Street, N.W., Washington, DC, 20005-3315, US

(21) Appl. No.: 10/218,914

(22) Filed: Aug. 15, 2002

Related U.S. Application Data
(63) Continuation-in-part of application No. 09/985,361, filed on Nov. 2, 2001.
Application No. 09/985,361 is a continuation of application No. 09/697,597, filed on Oct. 27, 2000, now Pat. No. 6,332,864.
Application No. 09/697,597 is a continuation of application No. 09/492,777, filed on Jan. 28, 2000, now Pat. No. 6,162,168.
Application No. 09/492,777 is a continuation of application No. 08/778,277, filed on Jan. 2, 1997, now Pat. No. 6,050,936.
(63) Continuation-in-part of application No. 10/138,520, filed on May 6, 2002.
Application No. 10/138,520 is a continuation of application No. 09/843,078, filed on Apr. 27, 2001, now Pat. No. 6,402,680.
Application No. 09/843,078 is a division of application No. 09/522,068, filed on Mar. 9, 2000, now Pat. No. 6,264,602.
Application No. 09/522,068 is a division of application No. 09/124,321, filed on Jul. 29, 1998, now Pat. No. 6,077,214.
(63) Continuation-in-part of application No. 10/136,440, filed on May 2, 2002.
Application No. 10/136,440 is a continuation of application No. 09/711,501, filed on Nov. 14, 2000, now Pat. No. 6,402,679.
Application No. 09/711,501 is a continuation of application No. 09/157,486, filed on Sep. 21, 1998, now Pat. No. 6,183,411.

(51) Int. Cl. A61N 1/362

(52) U.S. Cl. 600/16

(57) ABSTRACT

Devices for treating heart failure by reducing wall tension may be configured to be positioned external to the heart. Devices may also be configured to draw portions of walls of a heart chamber toward each other.

### RELATED APPLICATIONS

This application is a continuation-in-part of U.S. application Ser. No. 09/985,361, filed Nov. 2, 2001, which is a continuation of U.S. application Ser. No. 09/697,597, filed Oct. 27, 2000, now U.S. Pat. No. 6,332,864, which is a continuation of U.S. application Ser. No. 09/492,777, filed Jan. 28, 2000, now U.S. Pat. No. 6,162,168, which is a continuation of U.S. application Ser. No. 08/778,277, filed Jan. 2, 1997, now U.S. Pat. No. 6,050,936. This application also is a continuation-in-part of U.S. application Ser. No. 10/138,520, filed May 6, 2002, which is a continuation of U.S. application Ser. No. 09/843,078, filed Apr. 27, 2001, now U.S. Pat. No. 6,402,680, which is a divisional of U.S. application Ser. No. 09/522,068, filed Mar. 9, 2000, now U.S. Pat. No. 6,264,602, which is a divisional of U.S. application Ser. No. 09/124,321, filed Jul. 29, 1998, now U.S. Pat. No. 6,077,214. This application also is a continuation-in-part of U.S. application Ser. No. 10/136,440, filed May 2, 2002, which is a continuation of U.S. application Ser. No. 09/711,501, filed Nov. 14, 2000, now U.S. Pat. No. 6,402,679, which is a continuation of U.S. application Ser. No. 09/157,486, filed Sep. 21, 1998, now U.S. Pat. No. 6,183,411. The disclosures of all of the above-referenced applications and patents are incorporated herein by reference in their entirety.

### FIELD OF THE INVENTION

The present invention pertains to the field of apparatus for treatment of a failing heart. In particular, the apparatus of the present invention is directed toward reducing the wall stress in the failing heart.

### BACKGROUND OF THE INVENTION

The syndrome of heart failure is a common course for the progression of many forms of heart disease. Heart failure may be considered to be the condition in which an abnormality of cardiac function is responsible for the inability of the heart to pump blood at a rate commensurate with the requirements of the metabolizing tissues, or can do so only at an abnormally elevated filling pressure. There are many specific disease processes that can lead to heart failure with a resulting difference in pathophysiology of the failing heart, such as the dilatation of the left ventricular chamber. Etiologies that can lead to this form of failure include idiopathic cardiomyopathy, viral cardiomyopathy, and ischemic cardiomyopathy.

The process of ventricular dilatation is generally the result of chronic volume overload or specific damage to the myocardium. In a normal heart that is exposed to long term increased cardiac output requirements, for example, that of an athlete, there is an adaptive process of slight ventricular dilation and muscle myocyte hypertrophy. In this way, the heartfully compensates for the increased cardiac output requirements. With damage to the myocardium or chronic volume overload, however, there are increased requirements put on the contracting myocardium to such a level that this compensated state is never achieved and the heart continues to dilate.

The basic problem with a large dilated left ventricle is that there is a significant increase in wall tension and/or stress both during diastolic filling and during systolic contraction. In a normal heart, the adaptation of muscle hypertrophy (thickening) and ventricular dilatation maintain a fairly constant wall tension for systolic contraction. However, in a failing heart, the ongoing dilatation is greater than the hypertrophy and the result is a rising wall tension requirement for systolic contraction. This is felt to be an ongoing insult to the muscle myocyte resulting in further muscle damage. The increase in wall stress is also true for diastolic filling. Additionally, because of the lack of cardiac output, there is generally a rise in ventricular filling pressure from several physiologic mechanisms. Moreover, in diastole there is both a diameter increase and a pressure increase over normal, both contributing to higher wall stress levels. The increase in diastolic wall stress is felt to be the primary contributor to ongoing dilatation of the chamber.

Prior art treatments for heart failure fall into three generally categories. The first being pharmacological, for example, diuretics. The second being assist systems, for example, pumps. Finally, surgical treatments have been experimented with, which are described in more detail below.

With respect to pharmacological treatments, diuretics have been used to reduce the workload of the heart by reducing blood volume and preload. Clinically, preload is defined in several ways including left ventricular end diastolic pressure (LVEDP), or left ventricular end diastolic volume (LVEDV). Physiologically, the preferred definition is the length of stretch of the sarcomere at end diastole. Diuretics reduce extra cellular fluid which builds in congestive heart failure patients increasing preload conditions. Nitrates, arteriolar vasodilators, angiotensin converting enzyme inhibitors have been used to treat heart failure through the reduction of cardiac workload through the reduction of afterload. Afterload may be defined as the tension or stress required in the wall of the ventricle during ejection. Inotropes like digoxin are cardiac glycosides and function to increase cardiac output by increasing the force and speed of cardiac muscle contraction. These drug therapies offer some beneficial effects but do not stop the progression of the disease.

Assist devices include mechanical pumps and electrical stimulators. Mechanical pumps reduce the load on the heart by performing all or part of the pumping function normally done by the heart. Currently, mechanical pumps are used to sustain the patient while a donor heart for transplantation becomes available for the patient. Electrical stimulation such as bi-ventricular pacing have been investigated for the treatment of patients with dilated cardiomyopathy.

There are at least three surgical procedures for treatment of heart failure: 1) heart transplant; 2) dynamic cardiomyoplasty; and 3) the Batista partial left ventriculectomy. Heart transplantation has serious limitations including restricted availability of organs and adverse effects of immunosuppressive therapies required following heart transplantation. Cardiomyoplasty includes wrapping the heart with skeletal muscle and electrically stimulating the muscle to contract synchronously with the heart in order to help the pumping function of the heart. The Batista partial left ventriculectomy includes surgically remodeling the left ventricle by removing a segment of the muscular wall. This procedure reduces the diameter of the dilated heart, which in turn reduces the loading of the heart. However, this extremely invasive procedure reduces muscle mass of the heart.

### SUMMARY OF THE INVENTION

The present invention pertains to a non-pharmacological, passive apparatus for the treatment of a failing heart. The device is configured to reduce the tension in the heart wall. It is believed to reverse, stop or slow the disease process of a failing heart as it reduces the energy consumption of the failing heart, decrease in isovolumetric contraction, increases sarcomere shortening during contraction and an increase in isotonic shortening in turn increases stroke volume. The device reduces wall tension during diastole (preload) and systole.

In one embodiment, the apparatus includes a tension member for drawing at least two walls of the heart chamber toward each other to reduce the radius or area of the heart chamber in at least one cross sectional plane. The tension member has anchoring member disposed at opposite ends for engagement with the heart or chamber wall.

In another embodiment, the apparatus includes a compression member for drawing at least two walls of a heart chamber toward each other. In one embodiment, the compression member includes a balloon. In another embodiment of the apparatus, a frame is provided for supporting the compression member.

Yet another embodiment of the invention includes a clamp having two ends biased toward one another for drawing at least two walls of a heart chamber toward each other. The clamp includes at least two ends having atraumatic anchoring member disposed thereon for engagement with the heart or chamber wall.

### BRIEF DESCRIPTION OF THE DRAWINGS

FIG. 1 is a transverse cross-section of the left and right ventricles of a human heart showing the placement of a splint in accordance with the present invention;

FIG. 2 is a transverse cross-section of the left and right ventricles of a human heart showing the placement of a balloon device in accordance with the present invention;

FIG. 3 is a transverse cross-section of the left and right ventricles of a human heart showing the placement of an external compression frame structure in accordance with the present invention;

FIG. 4 is a transverse cross-section of the left and right ventricles of a human heart showing a clamp in accordance with the present invention;

FIG. 5 is a transverse cross-section of the left and right ventricles of a human heart showing a three tension member version of the splint of FIG. 1;

FIG. 6 is a transverse cross-section of the left and right ventricles of a human heart showing a four tension member version of the splint shown in FIG. 1;

FIG. 7 is a vertical cross-section of the left ventricle and atrium, the left ventricle having scar tissue;

FIG. 8 is a vertical cross-section of the heart of FIG. 7 showing the splint of FIG. 1 drawing the scar tissue toward the opposite wall of the left ventricle;

FIG. 9 is a vertical cross-section of the left ventricle and atrium of a human heart showing a version of the splint of FIG. 1 having an elongate anchor bar;

FIG. 10A is a vertical side view of a heart including a transventricular splint and band splint;

FIG. 10B is a horizontal cross section of the heart, splint and band splint of FIG. 10A;

FIG. 10C is a vertical view of a heart including a transventricular splint and a partial band splint;

FIG. 10D is a horizontal cross sectional view of the heart, splint and band splint of FIG. 10C;

FIG. 11 is a vertical view of the heart in phantom line including a band splint;

FIG. 12 is an alternate embodiment of the band splint of FIG. 11;

FIG. 13 is an alternate embodiment of the band splint of FIG. 11;

FIG. 14 is an alternate embodiment of the band splint of FIG. 11;

FIG. 15 is a vertical view of the heart including a mesh wrap;

FIG. 16 is a view of an alternate embodiment of a heart wall tension apparatus in accordance with the present invention;

FIG. 17 is a view of an alternate embodiment of a heart wall tension apparatus in accordance with the present invention;

FIG. 18 is a view of an alternate embodiment of a heart wall tension apparatus in accordance with the present invention;

FIG. 19 is a view of an alternate embodiment of a heart wall tension apparatus in accordance with the present invention;

FIG. 20 is a view of an alternate embodiment of a heart wall tension apparatus in accordance with the present invention;

FIG. 21 is a view of an alternate embodiment of a heart wall tension apparatus in accordance with the present invention;

FIG. 22 is a view of an alternate embodiment of a heart wall tension apparatus in accordance with the present invention;

FIG. 23 is a view of an alternate embodiment of a heart wall tension apparatus in accordance with the present invention;

FIG. 24 is a view of an alternate embodiment of a heart wall tension apparatus in accordance with the present invention;

FIG. 25 is a view of an alternate embodiment of a heart wall tension apparatus in accordance with the present invention;

FIG. 26 is a view of an alternate embodiment of a heart wall tension apparatus in accordance with the present invention;

FIG. 27 is a view of an alternate embodiment of a heart wall tension apparatus in accordance with the present invention;

FIG. 28A is a view of an alternate embodiment of a heart wall tension apparatus in accordance with the present invention;

FIG. 28B is a view of an alternate embodiment of a heart wall tension apparatus in accordance with the present invention;

FIG. 29 is a idealized cylindrical model of a left ventricle of a human heart;

FIG. 30 is a splinted model of the left ventricle of FIG. 29;

FIG. 31 is a transverse cross-sectional view of FIG. 30 showing various modeling parameters;

FIG. 32 is a transverse cross-section of the splinted left ventricle of FIG. 30 showing a hypothetical force distribution; and

FIG. 33 is a second transverse cross-sectional view of the model left ventricle of FIG. 30 showing a hypothetical force distribution.

### DETAILED DESCRIPTION OF THE INVENTION

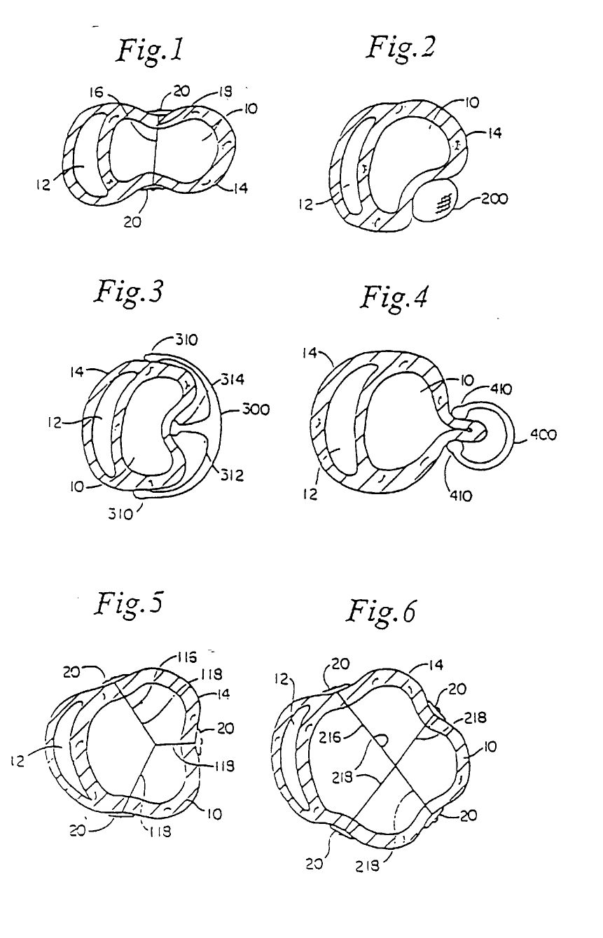

Referring now to the drawings wherein like reference numerals refer to like elements throughout the several views, FIG. 1 shows a transverse cross-section of a left ventricle 10 and a right ventricle 12 of a human heart 14. Extending through the left ventricle is a splint 16 including a tension member 18 and oppositely disposed anchors 20. Splint 16 as shown in FIG. 1 has been positioned to draw opposite walls of left ventricle 10 toward each other to reduce the “radius” of the left ventricular cross-section or the cross-sectional area thereof to reduce left ventricular wall stresses. It should be understood that although the splint 16 and the alternative devices disclosed herein are described in relation to the left ventricle of a human heart, these devices could also be used to reduce the radius or cross-sectional area of the other chambers of a human heart in transverse or vertical directions, or at an angle between the transverse and vertical.

FIG. 2 discloses an alternate embodiment of the present invention, wherein a balloon 200 is deployed adjacent the left ventricle. The size and degree of inflation of the balloon can be varied to reduce the radius or cross-sectional area of left ventricle 10 of heart 14.

FIG. 3 shows yet another alternative embodiment of the present invention deployed with respect to left ventricle 10 of human heart 14. Here a compression frame structure 300 is engaged with heart 14 at atraumatic anchor pads 310. A compression member 312 having an atraumatic surface 314 presses against a wall of left ventricle 10 to reduce the radius or cross-sectional area thereof.

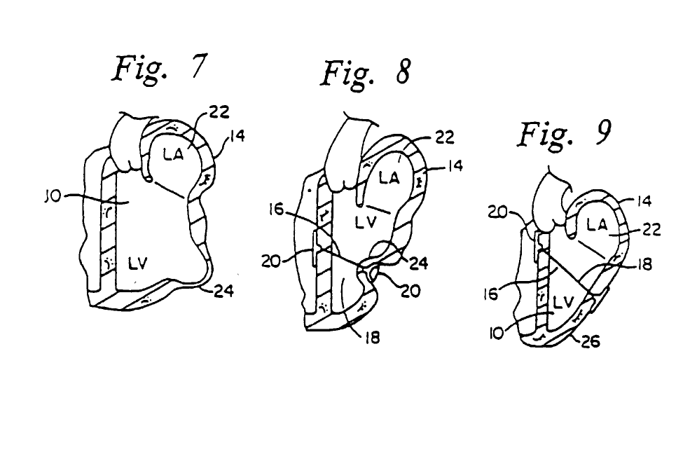

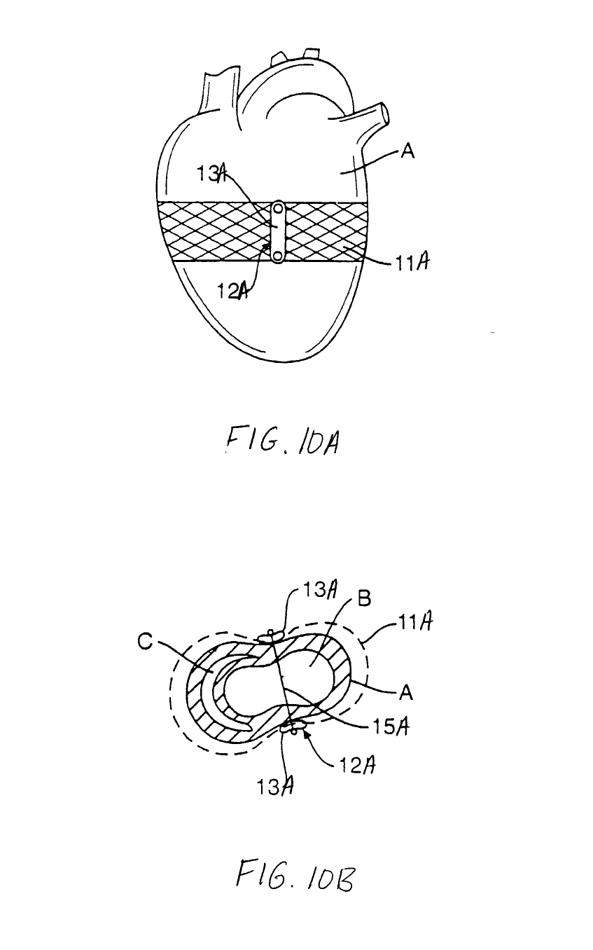

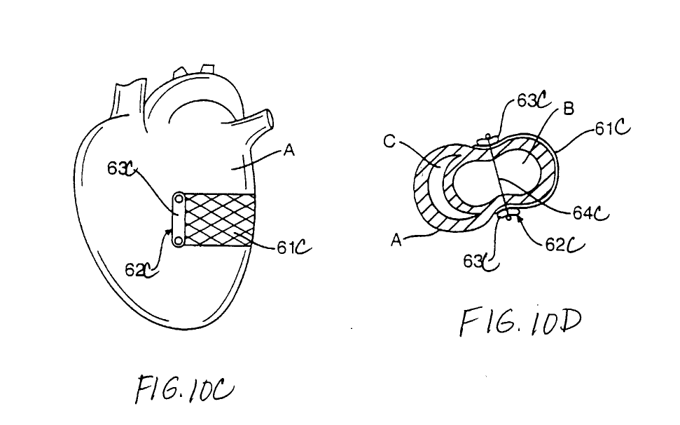

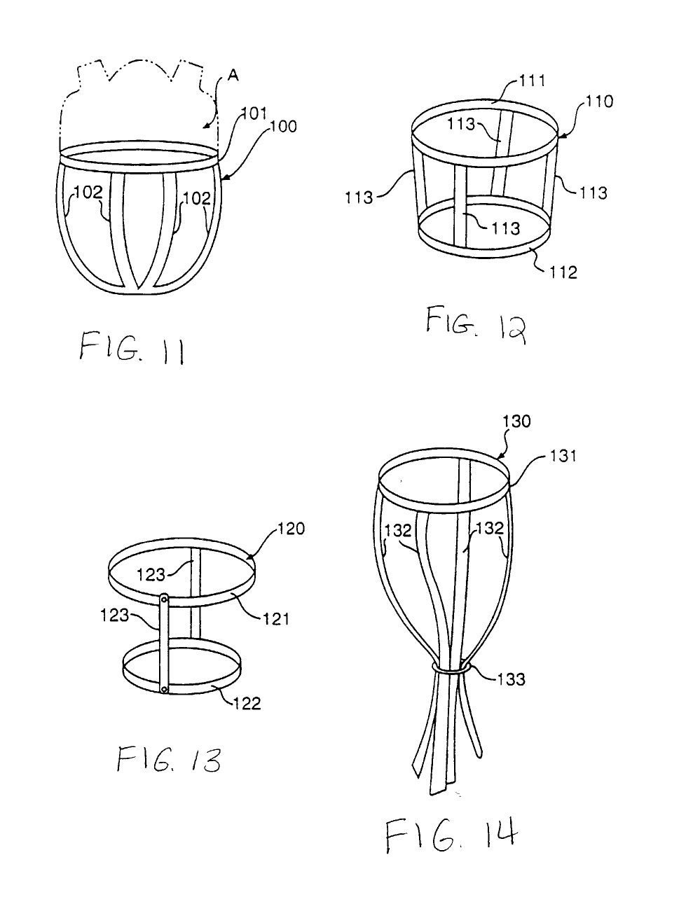

FIG. 4 is a transverse cross-sectional view of human heart 14 showing yet another embodiment of the present invention. In this case a clamp 400 having atraumatic anchor pads 410 biased toward each other is shown disposed on a wall of left ventricle 10. Here the radius or cross-sectional area of left ventricle 10 is reduced by clamping off the portion of the wall between pads 410. Pads 410 can be biased toward each other and/or can be held together by a locking device.

Each of the various embodiments of the present invention disclosed in FIGS. 1-4 can be made from materials which can remain implanted in the human body indefinitely. Such biocompatible materials are well-known to those skilled in the art of clinical medical devices.

FIG. 5 shows an alternate embodiment of the splint of FIG. 1 referred to in FIG. 5 by the numeral 116. The embodiment 116 shown in FIG. 5 includes three tension members 118 as opposed to a single tension member 18 as shown in FIG. 1. FIG. 6 shows yet another embodiment of the splint 216 having four tension members 218. It is anticipated that in some patients, the disease process of the failing heart may be so advanced that three, four or more tension members may be desirable to reduce the heart wall stresses more substantially than possible with a single tension member as shown in FIG. 1.

FIG. 7 is a partial vertical cross-section of human heart 14 showing left ventricle 10 and left atrium 22. As shown in FIG. 7, heart 14 includes a region of scar tissue 24 associated with an aneurysm or ischemia. As shown in FIG. 7, the scar tissue 24 increases the radius or cross-sectional area of left ventricle 10 in the region affected by the scar tissue. Such an increase in the radius or cross-sectional area of the left ventricle will result in greater wall stresses on the walls of the left ventricle.

FIG. 8 is a vertical cross-sectional view of the heart 14 as shown in FIG. 7, wherein a splint 16 has been placed to draw the scar tissue 24 toward an opposite wall of left ventricle 10. As a consequence of placing splint 16, the radius or cross-sectional area of the left ventricle affected by the scar tissue 24 is reduced. The reduction of this radius or cross-sectional area results in reduction in the wall stress in the left ventricular wall and thus improves heart pumping efficiency.

FIG. 9 is a vertical cross-sectional view of left ventricle 10 and left atrium 22 of heart 14 in which a splint 16 has been placed. As shown in FIG. 9, splint 16 includes an alternative anchor 26. The anchor 26 is preferably an elongate member having a length as shown in FIG. 9 substantially greater than its width (not shown). Anchor bar 26 might be used to reduce the radius or cross-sectional area of the left ventricle in an instance where there is generalized enlargement of left ventricle 10 such as in idiopathic dilated cardiomyopathy. In such an instance, bar anchor 26 can distribute forces more widely than anchor 20.

In use, the various embodiments of the present invention are placed in or adjacent the human heart to reduce the radius or cross-section area of at least one chamber of the heart. This is done to reduce wall stress or tension in the heart or chamber wall to slow, stop or reverse failure of the heart. In the case of the splint 16 shown in FIG. 1, a canula can be used to pierce both walls of the heart and one end of the splint can be advanced through the canula from one side of the heart to the opposite side where an anchor can be affixed or deployed. Likewise, an anchor is affixed or deployed at the opposite end of splint 16.

FIG. 10A is a view of a heart A in a normal, generally vertical orientation. A wrap 11A surrounds heart A and a transventricular splint 12A extends through the heart and includes an anchor or anchor pad 13A disposed on opposite sides of the heart. FIG. 10B is a horizontal cross sectional view of heart A taken through wrap 11A and splint 12A. Splint 12A includes a tension member 15A extending through left ventricle B. Anchor pads 13A are disposed at each end of tension member 15A. Right ventricle C is to the left of left ventricle B.

In FIG. 10A, wrap 11A and splint 12A are shown engaged with heart A. In FIG. 10B, heart A is shown spaced from wrap 11A except at anchor pads 13A. In FIG. 10B, heart A is thus at a point in the cardiac cycle where the muscles are shortening during systole, or have yet to stretch sufficiently during diastolic expansion to reach wrap 11A. Accordingly, wrap 11A can be considered a restrictive device as it does not engage the heart full cycle. Although wrap 11A is in contact with heart A at pads 13A, only the splint is providing a compressive force to change the shape of the heart and limiting the stress of the heart in FIG. 10B.

If heart A, as shown in FIG. 10B is at end systole, transventricular splint 12A is a full cycle device as the cross section of left ventricle B does not have the generally circular unsplinted shape. It can be appreciated that transventricular splint 12A can be used without wrap 11A. Alternately, wrap 11A could be secured to heart A by sutures or other means than splint 12A, in which case wrap 11A would be merely a restrictive device. It should be noted that unless wrap 11A extends vertically along heart A a sufficient amount, as heart A expands and engages wrap 11A, the portion of left ventricle B disposed above or below wrap 11A could expand substantially further than that portion of the left ventricle wall restrained by wrap 11A. In such a case, left ventricle B could have a bi-lobed shape in a vertical cross section. As such, the wrap 11A would not be merely limiting the size of the left ventricle, but rather inducing a shape change in the left ventricle. In such a case, the element 11A would not be a wrap, but rather a splint which could be referred to as a “band splint”.

Each of the splints, wraps and other devices disclosed in this application preferably do not substantially deform during the cardiac cycle such that the magnitude of the resistance to the expansion or contraction of the heart provided by these devices is reduced by substantial deflection. It is, however, contemplated that devices which deflect or elongate elastically under load are within the scope of the present invention, though not preferred. The materials from which each device are formed must be biocompatible and are preferably configured to be substantially atraumatic.

FIG. 10C is a vertical view of heart A, partial wrap 61 C and transventricular splint 62C. Transventricular splint 62C includes anchor pads 63C. FIG. 10D is a horizontal cross sectional view of heart A, partial band splint 61C and splint 62C. Splint 62C is essentially similar to wrap or band splint 12A shown in FIGS. 10A and 10B. Partial band splint 61C is also essentially similar to wrap or band splint 11A shown in FIGS. 10A and 10B except that band splint 61C only surrounds a portion of heart A. This portion is shown in FIGS. 10C and 10D to the left including a portion of left ventricle B.

FIG. 11 is a vertical view of heart A shown in phantom line. Shown disposed about the ventricles of heart A is a basket-like band splint 100. Band splint 100 includes a horizontal encircling band 101 around an upper region of the ventricles and four bands 102 which extend downward toward the apex of heart A. It can be appreciated that bands 102 can act as splints to form four lobes in heart A in a horizontal plane. Depending on the placement of bands 102 around heart A, lobes could be created only in the left ventricle or in the left ventricle and/or other chambers of the heart. Band 102 is joined at the apex Band 101 and band 102 can be made from a webbing, fabric or other biocompatible material.

If band splint 100 substantially elongated elastically under normal operating loads, it could be friction fit to heart A and act full cycle, limiting muscle stress at end diastole as well end systole. Band splint 100 could be sutured into place or otherwise held on heart A and act as a restrictive device. If band 101 were securely fastened to heart A, bands 102 could limit the vertical elongation of heart A during diastolic filling.

FIG. 12 is an alternate embodiment 110 of the band splint of FIG. 11. Band splint 110 includes a horizontally heart encircling band 111 and four bands 113 extending downward from band 111. Bands 113, however, unlike bands 102 of band splint 100 do not extend to the apex of heart A, but rather to a second horizontally heart encircling band 112.

Band splint 110 could be made of the same materials as band splint 100. Band splint 110 can also be used in a manner similar to band splint 100 except that band splint 110 would limit the vertical elongation of the ventricles less than band splint 100.

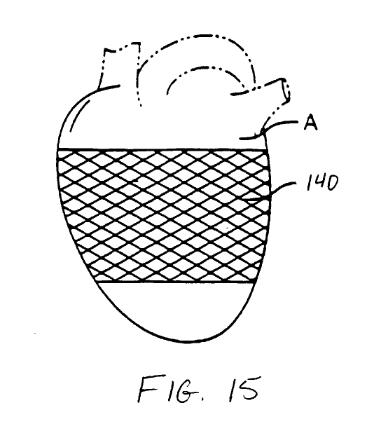

FIG. 13 is yet another alternate embodiment 120 of the wrap of FIG. 11. Band splint 120 closely resembles alternate embodiment 110 of FIG. 12, except that rather than having four vertically extending web members, band splint 120 includes two substantially rigid members 123 interconnecting two horizontally encircling web members 121 and 122.

FIG. 14 is yet another alternate embodiment 130 of the band splint of FIG. 11. Like the wrap of FIG. 11, band splint 130 includes a horizontally encircling member 131 and four downwardly extending members 132. At a location proximate of the apex of heart A, members 132 are joined by a ring 133. Members 132 extend through ring 133. Ring 133 can be used to adjust the length of members 132 between band 131 and ring 133. Ring 133 can be formed from metallic material and crimped inwardly to fix its position along members 132. Other means of holding ring 133 in position would be readily apparent to those skilled in the art.

FIG. 15 is a vertical view of heart A including a wrap 140. Wrap 140 extends vertically along the heart A to the apex. It can be appreciated that wrap 140 could be used as restrictive or full cycle device.

The devices and methods of the present invention can reduce heart wall stress throughout the cardiac cycle including end diastole and end systole. Alternatively, they can be used to reduce wall stress during the portions of the cardiac cycle not including end systole. Those devices which operate throughout the cardiac cycle are referred to herein as “full cycle splints”. Those devices which do not operate to reduce wall stress during end stage systole are referred to as “restrictive devices”. Restrictive devices include both “restrictive splints” which alter the geometric shape of the left ventricle, and “wraps” which merely limit the magnitude of the expansion of the left ventricle during diastolic filling without a substantial shape change.

Improving muscle shortening both total length change and extent at end systole, is particularly important in symptomatic heart failure wherein the heart has decreased left ventricle function and has enlarged. Full cycle splinting can be used to obtain a substantial increase in muscle shortening. Improved shortening will lead to an increase in pump function, and chronically may result in muscle strengthening and reversal of the disease because of increased pumping efficiency. The increase in shortening should be balanced against a reduction in chamber volume.

In asymptomatic, early stage heart failure, it may be possible to use only a restrictive device or method as elevated wall stress is considered to be an initiator of muscle damage and chamber enlargement. Restrictive devices and methods acting during diastole will reduce the maximum wall stress experience during end diastole and early systole. It should be understood that restrictive devices and methods can be used in combination with full cycle splinting to more precisely control or manipulate stress reduction throughout the cardiac cycle.

The magnitude of shape change in the case of full cycle splinting becomes very important as full cycle splinting generally reduces chamber volume more than restrictive splinting. Although as with restrictive devices, the type of shape change is also important to allow for variable preload strain. Both restrictive device and full cycle splints reduce chamber volume as they reduce the cross sectional area of the chamber during the cardiac cycle. The magnitude of the shape change can vary from very slight at end diastole, such that chamber volume is only slightly reduced from the unsplinted end diastolic volume, to an extreme reduction in volume, for example, complete bifurcation by transventricular splint. The magnitude of the shape change, for example, as measured by the ratio of splint length to non-splinted ventricular diameter, is preferably modulated to reduce muscle stress while not overly reducing chamber volume. For full cycle splint, the reduction of chamber volume is compensated for by increased contractile shortening, which in turn leads to an increased ejection fraction, i.e., the ratio of the stroke volume to chamber volume. For given stress/volume and stress/shortening relationships, there will be a theoretical optimum maximal stroke volume. Clinically, 20% to 30% stress reduction is expected to be attainable through full cycle bi-lobe splinting. For additional information regarding full cycle splinting and restrictive splinting, see U.S. Pat. No. 6,077,214, the complete disclosure of which is incorporated by reference herein.

The splints, wraps, and other devices of FIGS. 10A-15 preferably do not substantially deform during the cardiac cycle such that the magnitude of the resistance to the expansion or contraction of the heart provided by these devices is reduced by substantial deflection. It is, however, contemplated that devices which deflect or elongate elastically under load are within the scope of the present invention, though not preferred. The materials from which each device are formed must be biocompatible and are preferably configured to be substantially atraumatic.

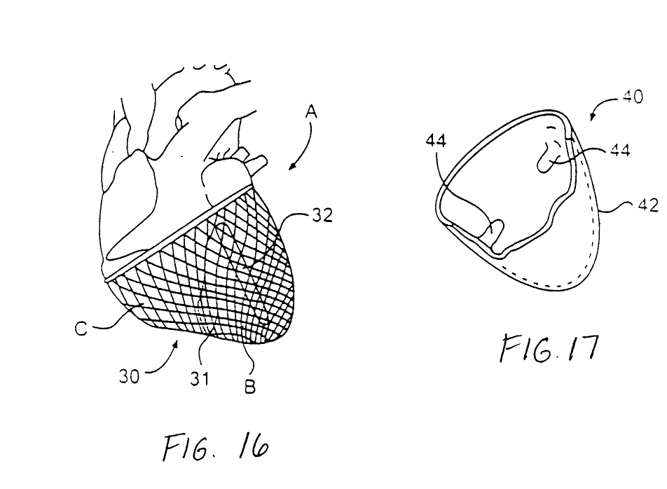

FIG. 16 is an alternate embodiment of a heart wall stress reduction device 30 disposed on heart A which is shown in a generally vertical orientation. Device 30 preferably includes a sock 31 formed from a porous mesh of biocompatible fabric such as polyester. Sock 31 preferably does not substantially stretch or elongate under operational loads. Sock 31 could, however, be made from a material which deforms elastically at operational loads. Disposed between sock 31 and heart A is an elongate bar 32. Bar 32 is preferably held against left ventricle B with sufficient force to create a shape change of the heart when a second bar 32 is disposed between sock 31 and the posterior side of heart A. Sock 31 is preferably held in place on heart A by sutures.

FIG. 17 is yet another alternate embodiment 40 of a heart wall stress reduction device. Device 40 is similar to device 30 except that it includes a shell 42 which is substantially rigid under operational loads rather than a sock 31 and inwardly protruding members 40 rather than a bar 32. Shell 42 can be slipped over heart A to create a shape change similar to that shown in FIG. 1. Members 44 are thus preferably profiled such that they can be slid atraumatically over heart A to place device 40.

Device 40 is preferably made from a biocompatible metal or plastic. The material is preferably relatively light materials to enhance stability of the device on heart A. Light metals which could be used to form device include Co—Cr—Mo alloys, Co—Ni—Cr—Mo alloy (MP35N), carbon and titanium alloys (Ti-6AL-4V). In addition to plastics such as polyester, device 40 could be formed from composites such as carbon fibers/epoxy, polyester/epoxy, or amide fiber/epoxy, for example. The surface of protrusions 44 preferably include a surface which promotes tissue ingrowth as described above. Device 40 can be held in place on heart A by sutures placed through apertures (not shown) in shell 42.

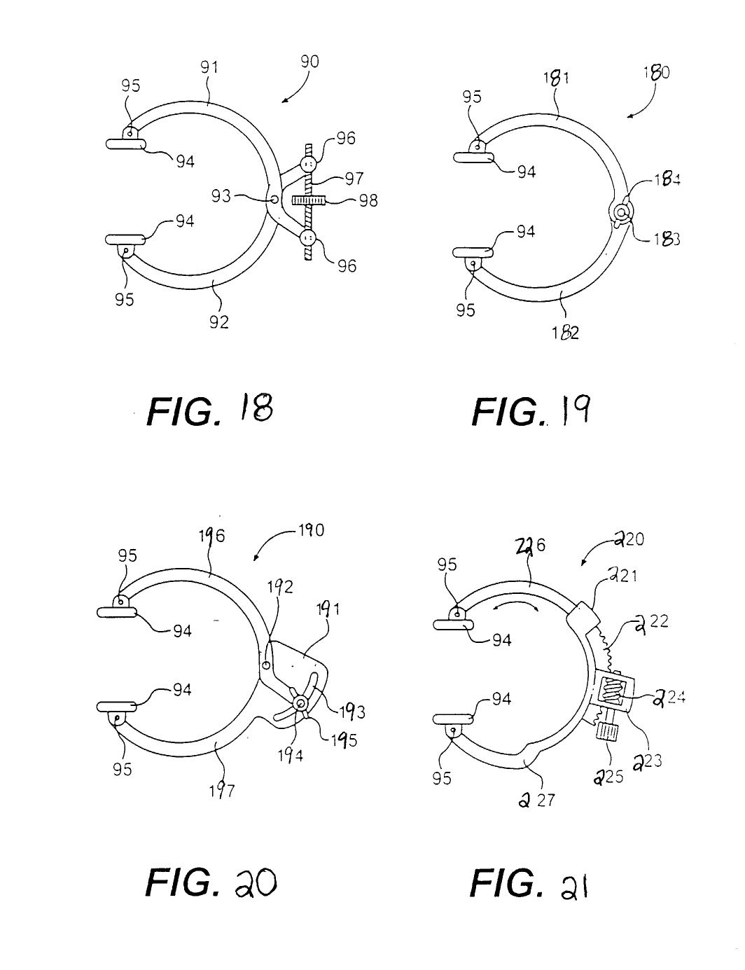

As an alternative embodiment, a device is provided that can be adjusted or sized prior to placement on heart A, devices such as those shown in FIGS. 18-27 can readily be adjusted in place on the heart. The devices of FIGS. 18-27 include mechanical mechanisms for adjusting anchor spacing. Each of these devices could be positioned in heart A to create a shape change similar to that of FIG. 1. The devices of FIGS. 18-27 are preferably made from light biocompatible metal and/or plastics. The anchors or pads preferably have a porous heart engaging surface to promote tissue ingrowth.

FIG. 18 is a view of yet another alternate embodiment of a heart wall stress reduction device 90 in accordance with the present invention. Device 90 includes two oppositely disposed arms 91 and 92 pivotally attached by a pin 93 to form a C-shape. Disposed at the free ends of each arm 91 and 92 is an anchor or anchor pad 94 pivotally attached to arms 91 and 92 by pins 95. Pivotally attached to the opposite ends of arms 91 and 92 are internally threaded members 96 into which is threaded a rod 97. Disposed along, and fixably attached to rod 97 is a thumb wheel 98 for rotating rod 97. Rod 97 is preferably flexible enough that as it is rotated to draw the ends of arms 91 and 92 together, it can be deformed such that wheel 98 will move to the right as upper member 96 pivots counterclockwise and lower member 96 pivots clockwise.

FIG. 19 is a view of yet an alternate embodiment 180 of a C-shaped heart wall stress reduction device. Device 180 includes arms 181 and 182. Disposed at the free ends of arms 181 and 182 are pads 94 pivotally connected thereto by pins 95. At the opposite ends of arms 181 and 182, they are joined by a bolt 183 and wing nut 184. Wing nut 184, when loosened will allow arms 181 and 182 to pivot around bolt 183. Wing nut 184 can be tightened to fix the relative position of arms 181 and 182 when the desired spacing of pads 94 has been achieved.

FIG. 20 is a view of yet an alternate embodiment 190 of a C-shaped heart wall stress reduction device. Device 190 is similar to device 190 except that oppositely disposed arms 196 and 197 are cantilevered beyond their pivotable attachment point at pin 192 to a bolt 194 and a wing nut 195. Arm 197 includes a plate 191 having an arc-like aperture 193 formed therein. Bolt 194 extends through aperture 193 and arm 196 such that when wing nut 195 is loose, bolt 194 can slide in aperture 193 to rotate arm 196 about pin 192 to adjust the spacing between pads 94. When the desired spacing is achieved, wing nut 195 can be tightened to fix the relative position of arms 196 and 197.

FIG. 21 is a view of yet another alternate embodiment of a generally C-shaped heart wall stress reduction device 220. Device 220 includes two oppositely disposed arms 226 and 227. Pads 94 are pivotally attached by pins 95 to the free ends of arms 226 and 227. The opposite end of arm 226 is slidably disposed through a receiving housing 221 at the opposite end of arm 227. The end of arm 227 extending through housing 221 includes teeth 222. Disposed between housing 221 and pad 94 and along arm 227 is a screw gear housing 223 which positions the threads of a screw gear 224 between teeth 222. Gear 224 includes a shaft having a thumb knob 225 attached thereto. Knob 225 can be used to rotate screw 224 to engage successive teeth 222 to move arm 226 relative to arm 227 in the directions shown by the arrow. Thus, in this manner, arm 226 can be moved to adjust the spacing between pads 94.

FIG. 22 shows yet another alternate embodiment of a generally C-shaped heart wall stress reduction device 230 in accordance with the present invention. Device 230 is similar to device 180 except for oppositely disposed arms 234 and 235 are pivotable about pin 231 and fixable in position by ratchet teeth 232 of arm 234 and an elongate member 233 connected to arm 235. Ratchet teeth are sloped such that as arm 234 is pivoted about pin 231 to bring pads 94 closer together, member 233 rides over successive teeth 232. If, however, it is attempted to rotate 234 in the opposite direction, teeth 232 are sloped to engage member 233 and resist the rotation of arm 234 about pin 231. Member 233 can be pulled away from teeth 232 to allow arm 234 to be pivoted in a clockwise direction.

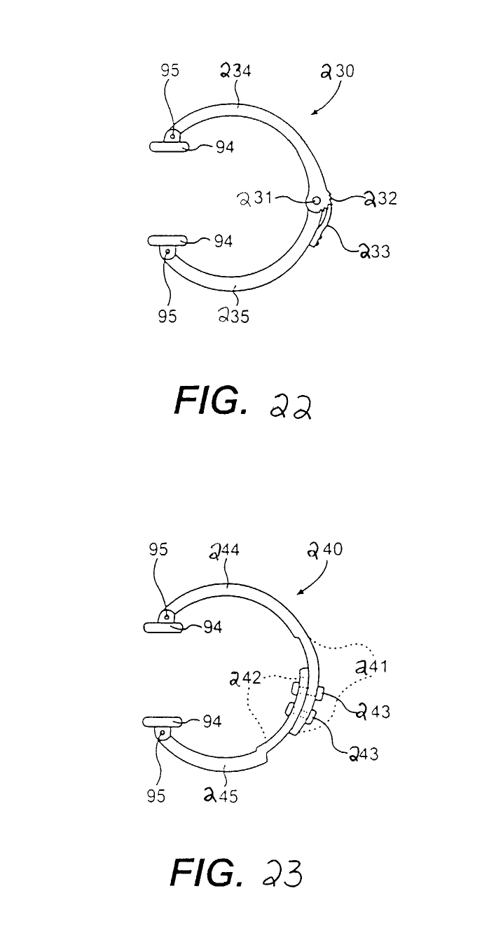

FIG. 23 is a view of yet another alternate embodiment of a generally C-shaped heart wall tension reduction device 240 in accordance with the present invention. Device 240 includes oppositely disposed arms 244 and 245. Anchors 94 are pivotally attached by pins 95 to the free ends of arms 244 and 245. The opposite ends of arms 244 and 245 include slots 241 and 242. As shown in FIG. 23, where slots 241 and 242 overlap, nut and bolt assemblies 243 are disposed therethrough. As can be appreciated, if nut and bolt assemblies 143 are loosened they will be free to slide within slots 241 and 242 such that the ends of arms 244 and 245 disposed opposite pads 94 can be slid over each other to adjust the distance between pads 94. Once the desired distance between pads 94 is obtained, nut and bolt assemblies can be tightened to fix the relative position of arms 244 and 245.

FIG. 24 is a view of yet an alternate embodiment of a generally C-shaped heart wall stress reduction device 250 in accordance with the present invention. Device 250 includes two oppositely disposed arms 253 and 254. Pads 94 are pivotally attached by pins 95 to the pins of arms 253 and 254. The opposite end of arm 253 is slidably received within an aperture of a receiving housing 251 connected to the opposite end of arm 254. A set screw 252 is threaded into housing 251 such that when set screw 252 is loose, arm 253 can slide within housing 251 to vary the distance between pads 94. Once the desired distance between pads 94 has been obtained, set screw 252 can be tightened to engage arm 253 and fix its position relative to arm 254.

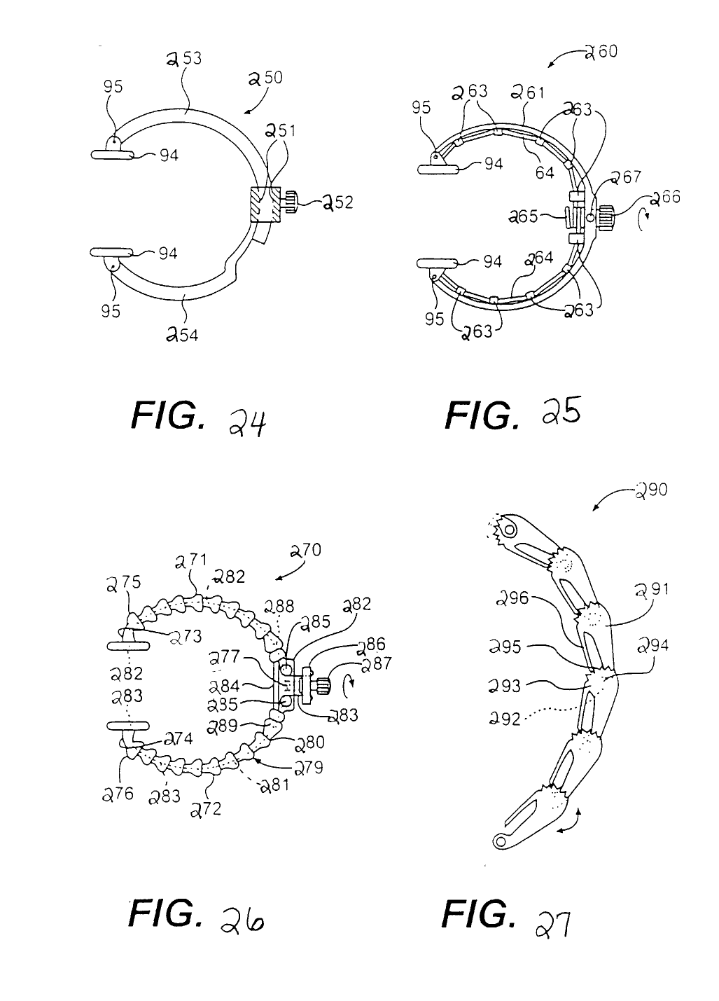

FIG. 25 is a view of yet an alternate generally C-shaped heart wall stress reduction apparatus 260 in accordance with the present invention. Device 260 includes a generally C-shaped arm 261 which has two oppositely disposed free ends. Pads 94 are pivotally connected by pins 95 to each of the free ends. Disposed along the interior arc of arm 261 are eyelets 263. Disposed through eyelets 263 is a line or cable 264 having two oppositely disposed ends fixably attached to opposite pads 94. A more centrally located portion of line 264 is at least partially wrapped around a spool 265. Spool 265 is rotatably connected to a generally central portion of member 261. A knob 266 is connected to spool 265 to allow rotation thereof. It can be appreciated that if spool 265 is rotated into the paper in the direction of the arrow, that the spacing between pads 94 will decrease as line 264 is pulled through eyelets 263 toward spool 265. It can be appreciated that if spool 265 is rotated in an opposite direction, pads 94 will move apart to the extent that member 261 is biased to expand outwardly. The position of spool 265 can be fixed when the desired spacing of pads 94 is obtained by tightening a set screw 267 disposed adjacent knob 266.

FIG. 26 is a view of yet an alternate embodiment of a generally C-shaped heart wall tension apparatus 270. Heart wall tension reduction apparatus 270 includes two oppositely disposed arms 271 and 272. Disposed at the free end of arms 271 and 272 are anchors 273 and 274, respectively. Anchors 273 and 274 can be anchor pads each having a disc-like heart engaging surface similar to that of anchor 94. The portion of anchors 273 and 274 opposite the disc-shaped portion includes socket shaped portions 275 and 276, respectively. These socket shaped portions 275 and 276 are shaped similarly to that of the socket portions of ball and socket joints. Disposed along the length of arms 271 and 272 are ball and socket members 279. Each member 279 includes a generally ball shaped or hemispherical end 281 and a complimentary concaved socket end 280. As shown, a series of members 279 are placed ball end to socket end to form each arm 271 and 272. The final ball end 281 of each arm 271 and 272 is disposed within sockets 275 and 276 respectively of anchors 273 and 274, respectively.

Each member 279 includes a longitudinal lumen extending therethrough. A line 282 extends through successive of these lumens in arms 271. A line 283 extends through arm 272 in a similar fashion. Lines 282 and 283 are free to move within the lumens but are fixably attached at their ends to anchors 273 and 274, respectively. The opposite ends of lines 282 and 283 pass over pulleys 285 and are connected to a spool or takeout reel 286 which in turn is pivotally connected to a central housing 284. Housing 284 includes oppositely disposed ball portions 288 and 289, which engage the sockets of the adjacent members 279. A knob 287 is provided to rotate spool 286. If spool 286 is rotated in the direction shown by the arrow, lines 282 and 283 will be drawn toward spool 286, which in turn will draw the adjacent ball and socket ends toward each other. When the force exerted by lines 282 and 283 is sufficient, friction between adjacent ball and socket ends will hold arms 271 and 272 in any position in which they have been placed. Thus, when the desired spacing between anchors 273 and 274 is obtained and lines 282 and 283 tightened, a set screw 277 can be tightened to retain spool 286 in position to maintain the spacing between anchors 173 and 174. Not only can the spacing between anchors 273 and 274 be controlled in this manner, but the shape of the arm can be altered along its length to be straight or arcuate to conform to the shape of the heart.

FIG. 27 is a view of an alternate arm configuration 290 which could be used in a generally C-shaped heart wall stress reduction apparatus. The principle of its operation would be similar to that of the device of FIG. 24, except that a plurality rather than one ratcheting member would be provided. By providing a plurality of ratcheting members, the shape of the arm can be altered along its length to be relatively straighter, or more arcuate depending upon the degree to which the various members are ratcheted with respect to each other.

Arm 290 includes a plurality of ratcheting members 291. A first end 292 of each member 291 is pivotally connected to the opposite end 293 of each member 291 by a pin 294. Each member can be rotated about pins 294 in the direction shown by the arrows. Teeth 295 are disposed at each end 293 to engage a ratcheting arm 296 extending from end 293 toward end 292. It can be appreciated that member 296 should be flexible enough that a physician can ratchet arm 296 over teeth 295 until the desired rotational position is obtained. The arms should also, however, be rigid enough that during normal operational heart loadings, member 291 remains between the teeth 295 selected by the physician.

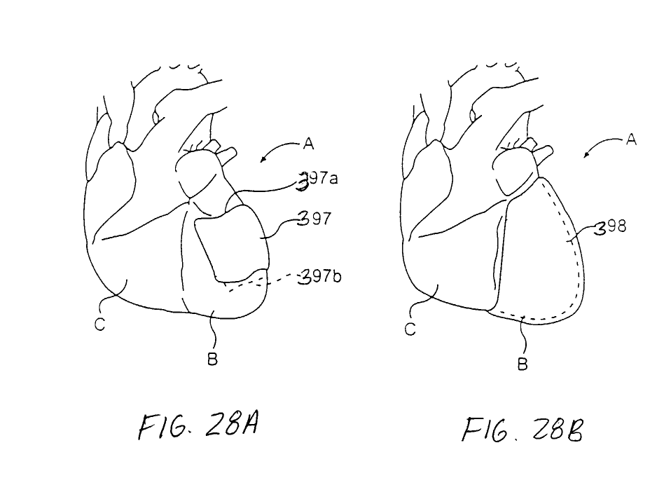

FIG. 28A is a generally vertical view of heart A. Yet another alternate embodiment of a heart wall stress reduction device 397 is shown on left ventricle B. Device 397 is preferably a sheet which has been wrapped around a portion of left ventricle B. The sheet includes a generally vertical elongate concave trough 397a on the anterior side of left ventricle B and a similar trough 397b on the posterior side of left ventricle B. The base of the trough can be made to engage opposite sides of the ventricle to create a bi-lobe shape.

The sheet is preferably formed in place on heart A to create the troughs 397a and 397b. The sheet can be formed from an epoxy or a composite including two or more of the following: epoxy, Dacron. TM., silicone or UV curable adhesive. The sheet, if made using a curable adhesive or epoxy should be placed prior to curing such that the sheet can be readily formed in a shape similar to that shown in FIG. 28A. During the curing process, the sheet can be held in place using one or more generally C-shaped heart wall tension reduction devices such as those shown in FIGS. 18-27.

The sheet material used to form device 397 could also be a malleable metal such as stainless steel. If a metal such as stainless steel were used to form the sheet, it could be bent to form a shape similar to that shown in FIG. 28A prior to placement on the heart or while being placed on heart A.

FIG. 28B is a generally vertical view of a heart A. Yet another embodiment of a heart wall stress reduction device 398 is shown disposed on left ventricle B. As shown in FIG. 28B, device 398 has a shell or helmet shape which substantially surrounds left ventricle B. Device 398 could be formed from materials including a malleable metal such as stainless steel that can be formed into shape prior to or during placement on the heart.

As shown herein the various heart wall stress reduction devices and methods of FIGS. 15-28B have been applied to form a bi-lobe configuration of the left ventricle. It can be appreciated that the devices and methods disclosed herein can also be used to create three or more lobes in the left ventricle. Additionally, the heart wall stress reduction devices and methods disclosed herein can also be used to change the shape of the remaining chambers of the heart in addition to the left ventricle. The external device as disclosed herein could also be used in conjunction with transventricular heart wall stress reduction devices. In such instance, both devices could be full cycle, restrictive, or one of the devices could be full cycle and the other restrictive. It can also be appreciated that the rotational positioning of the device about the heart can be varied to create a shape change between posterior and anterior anchors or between lateral anchors.

FIG. 29 is a view of a cylinder or idealized heart chamber 48 which is used to illustrate the reduction of wall stress in a heart chamber as a result of deployment of the splint in accordance with the present invention. The model used herein and the calculations related to this model are intended merely to illustrate the mechanism by which wall stress is reduced in the heart chamber. No effort is made herein to quantify the actual reduction which would be realized in any particular in vivo application.

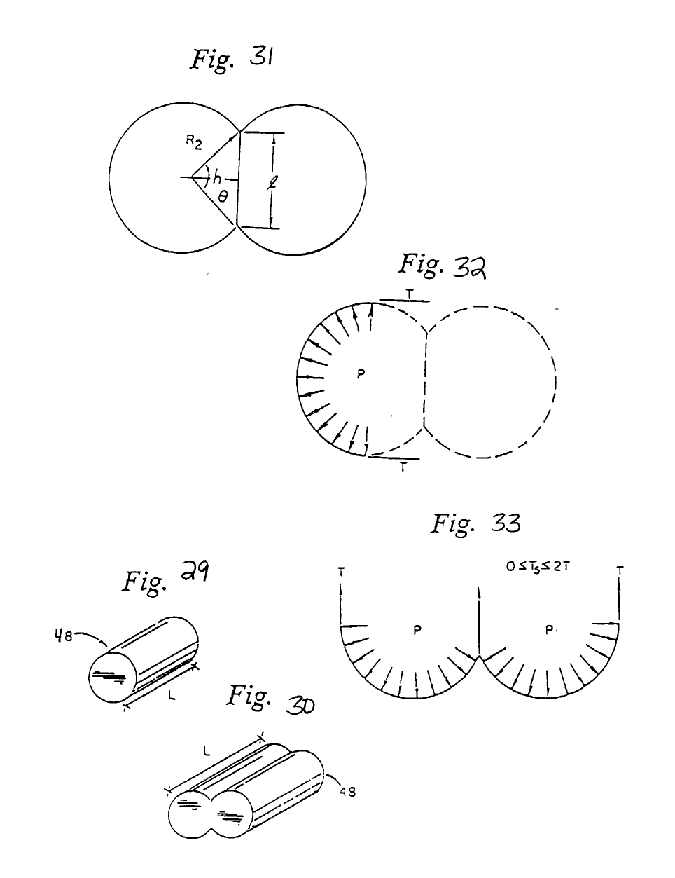

FIG. 30 is a view of the idealized heart chamber 48 of FIG. 29 wherein the chamber has been splinted along its length L such that a “figure eight” cross-section has been formed along the length thereof. It should be noted that the perimeter of the circular transverse cross-section of the chamber in FIG. 29 is equal to the perimeter of the figure eight transverse cross-section of FIG. 30. For purposes of this model, opposite lobes of the figure in cross-section are assumed to be mirror images.

FIG. 31 shows various parameters of the “figure eight” cross-section of the splinted idealized heart chamber of FIG. 30. Where is the length of the splint between opposite walls of the chamber, R is the radius of each lobe, Θ is the angle between the two radii of one lobe which extends to opposite ends of the portion of the splint within chamber 48 and h is the height of the triangle formed by the two radii and the portion of the splint within the chamber 48 (R is the radius of the cylinder of FIG. 29). These various parameters are related as follows:

h=R₂ COS(Θ/2)

λ=2R₂ SIN(Θ/2)

R₂=R₁ π/(2π−Θ)

From these relationships, the area of the figure eight cross-section can be calculated by:

A₂=2π(R₂)²(1−Θ/2π)+^{hλ}

Where chamber 48 is unsplinted as shown in FIG. 29 A₁, the original cross-sectional area of the cylinder is equal to A₂ where Θ=180.degree., h=0 and λ=2R₂ Volume equals A₂ times length L and circumferential wall tension equals pressure within the chamber times R₂ times the length L of the chamber.

Thus, for example, with an original cylindrical radius of four centimeters and a pressure within the chamber of 140 mm of mercury, the wall tension T in the walls of the cylinder is 104.4 newtons. When a 3.84 cm splint is placed as shown in FIGS. 30 and 16 such that λ=3.84 cm, the wall tension T is 77.33 newtons.

FIGS. 32 and 33 show a hypothetical distribution of wall tension T and pressure P for the figure eight cross-section. As Θ goes from 180.degree. to 0.degree., tension Tₛ in the splint goes from 0 to a 2T load where the chamber walls carry a T load.

It will be understood that this disclosure, in many respects, is only illustrative. Changes may be made in details, particularly in matters of shape, size, material, and arrangement of parts without exceeding the scope of the invention. Although the primary focus of the discussion of the devices and methods of the present invention herein relates to heart failure and the left ventricle, these devices and methods could be used to reduce stress in the heart's other chambers. Accordingly, the scope of the invention is as defined in the language of the appended claims.

What is claimed is:

1. An apparatus for reducing the stress in the wall of a heart chamber in at least one cross sectional plane, comprising:
means for passively changing the geometric shape of the chamber wall to change the internal shape of the chamber to reduce wall stress; and
means for engagement with the chamber wall coupled to the means for passively changing the internal shape of the wall.
